(19) United States
(12) Patent Application Publication
(10) Pub. No.: US 2025/0352043 A1
(43) Pub. Date: Nov. 20, 2025

(54) MEDICAL SYSTEMS AND DEVICES FOR THE AEROSOLIZATION OF A FLUID ENDOSCOPICALLY

(71) Applicant: Boston Scientific Scimed, Inc., Maple Grove, MN (US)

(72) Inventors: Scott Edward CORBEIL, Litchfield, NH (US); Rosangel Aimee RAMOS ESPINOZA, Boston, MA (US)

(73) Assignee: Boston Scientific Scimed, Inc., Maple Grove, MN (US)

(21) Appl. No.: 19/206,786

(22) Filed: May 13, 2025

Related U.S. Application Data
(60) Provisional application No. 63/647,337, filed on May 14, 2024.

(51) Int. Cl. A61B 1/00; A61B 1/005; A61B 1/015

(52) U.S. Cl. CPC A61B 1/00137; A61B 1/0008; A61B 1/00128; A61B 1/0051; A61B 1/015

(57) ABSTRACT

A medical device includes a sheath, a cap coupled to a distal end of the sheath, and a connector at a proximal end of the sheath. The sheath has a first lumen and a second lumen extending from the proximal end to the distal end of the sheath. The cap defines a cavity between the distal end of the sheath and a distal wall of the cap. A distal opening extends through the distal wall of the cap. The connector is configured to provide fluid communication between: (1) a first fluid source and the first lumen, and (2) a second fluid source and the second lumen. Upon delivery of a fluid from the first lumen into the cavity and a gas from the second lumen into the cavity, an at least partially aerosolized fluid is delivered via the distal opening.

### CROSS-REFERENCE TO RELATED APPLICATIONS

This application claims the benefit of priority to U.S. Provisional Application No. 63/647,337, filed on May 14, 2024, which is incorporated by reference herein in its entirety.

### TECHNICAL FIELD

This disclosure relates generally to medical systems and devices. In particular, this disclosure is directed to systems, devices, and methods for the aerosolization of one or more fluids endoscopically from a distal end of a device.

### BACKGROUND

One or more fluids, or agents, may be delivered to a treatment site during medical procedures, such as endoscopic procedures. For example, during an endoscopic procedure, a user may insert a portion of a device into a body lumen of a patient. A proximal end of the device may include a handle, or gripping portion, having one or more actuators, for example, to control (e.g., deflect and/or position) a distal end of the device and/or to control the delivery of fluid(s) from the distal end of the device during the procedure. The proximal end of the device may also include one or more ports in fluid communication with one or more lumens of the device. In aspects, fluid(s) may be delivered through the lumen(s) (e.g., via the one or more ports) to provide treatment at the treatment site near a distal end of the device. The treatment site may be internal to the patient, and thus may be remote from the user.

### SUMMARY

Aspects of the disclosure relate to, among other things, systems and devices configured to at least partially aerosolize one or more agents endoscopically. Each of the aspects disclosed herein may include one or more of the features described in connection with any of the other disclosed aspects.

According to an example, a medical device may include a sheath, a cap coupled to a distal end of the sheath, and a connector at a proximal end of the sheath. The sheath may have a first lumen and a second lumen extending from the proximal end to the distal end of the sheath. The cap may define a cavity between the distal end of the sheath and a distal wall of the cap. A distal opening may extend through the distal wall of the cap. The connector may be configured to provide fluid communication between: (1) a first fluid source and the first lumen, and (2) a second fluid source and the second lumen. Upon delivery of a fluid from the first lumen into the cavity and a gas from the second lumen into the cavity, an at least partially aerosolized fluid may be delivered via the distal opening.

Any of the devices disclosed herein may include any of the following features, additionally or alternatively, in any combination.

The distal opening of the cap may be aligned with a distal end of the second lumen. The cap may be comprised of a first cap portion and a second cap portion. The first cap portion may be configured to receive at least a portion of the second cap portion. The second cap portion may include at least one flange extending radially outward. The at least one flange may be configured to prevent proximal movement of the first cap portion.

The distal opening may be a first distal opening that extends through the distal wall of the first cap portion. The second cap portion may define a second distal opening that may be proximal of the first distal opening. A diameter of the second distal opening may be smaller than a diameter of the first distal opening.

A control member may extend proximally from a proximal end of the first cap portion. The sheath may be an inner sheath disposed within a lumen of an outer sheath. The control member may extend proximally into the lumen of the outer sheath.

The fluid may be a hemostatic agent. The gas may be at least one of carbon dioxide, oxygen, nitrogen, or atmospheric air.

A plurality of threads may be disposed on a distal end of the sheath. The cap may include a plurality of receiving threads configured to receive the plurality of threads of the sheath to secure the cap to the sheath. Rotation of the cap in a first direction relative to the sheath may increase a volume of the cavity. Rotation of the cap in a second direction relative to the sheath may decrease the volume of the cavity. The second direction may be opposite to the first direction.

A distal end of the first lumen may be distal relative to a distal end of the second lumen. A portion of the distal wall of the cap may be spaced away from and aligned with a distal opening of the first lumen. A cross-sectional shape of each of the first lumen and the second lumen may be C-shaped. The sheath may further define a third lumen disposed between the first lumen and the second lumen. A diameter of the cap may be larger than a diameter of the sheath.

According to another example, an insertion portion of a medical device may include a sheath defining a first lumen and a second lumen. The medical device may also include a cap. The cap may be configured to be coupled to a distal end of the sheath. The cap may include a distal wall having a distal opening extending therethrough. A cavity may be defined between the distal wall and a distal end of each of the first lumen and the second lumen. Upon delivery of a fluid from the first lumen into the cavity and upon delivery of a gas from the second lumen into the cavity, an aerosol may be delivered via the distal opening of the cap.

Any of the devices disclosed herein may include any of the following features, additionally or alternatively, in any combination.

The distal opening of the cap may be at least partially aligned with a distal opening of the second lumen. Rotation of the cap relative to the sheath in a first direction may be configured to increase a volume of the cavity. Rotation of the cap relative to the sheath in a second direction may be configured to decrease a volume of the cavity. The second direction may be opposite to the first direction.

According to another example, a medical device may include a sheath, a connector, and a cap. The sheath may define a first lumen and a second lumen. The connector may be fixed to a proximal end of the sheath. The connector may be fluidly connected to the first lumen and the second lumen. The cap may be fixed to a distal end of the sheath. The cap may define a cavity and a distal opening. The cap may be coupled to a distal end of the sheath. The distal opening may be at least partially aligned with a distal end of the second lumen. Upon (1) delivery of a fluid through the first lumen and into the cavity and (2) delivery of a gas through the second lumen and into the cavity, an aerosol may be delivered through the distal opening of the cap.

Any of the devices disclosed herein may include the following features, additionally or alternatively, in any combination.

Rotation of the cap relative to the sheath in a first direction may be configured to increase a volume of the cavity. Rotation of the cap relative to the sheath in a second direction may be configured to decrease a volume of the cavity. The second direction may be opposite to the first direction.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by various aspects of the elements and combinations particularly pointed out in the appended claims

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated herein and constitute a part of this specification, illustrate exemplary aspects of the disclosure and, together with the description, explain the principles of the disclosure.

FIG. 1 depicts a perspective view of a medical system, according to aspects of this disclosure.

FIG. 2A depicts a perspective view of a medical device, according to aspects of this disclosure.

FIGS. 2B, 2C, and 2D depict various cross-sectional views of various portions of a distal end of the medical device of FIG. 2A, according to aspects of this disclosure.

FIG. 3A depicts a perspective view of another medical device, according to aspects of this disclosure.

FIGS. 3B and 3C depict cross-sectional views of various portions of a distal end of the medical device of FIG. 3A, according to aspects of this disclosure.

FIG. 4 depicts a cross-sectional view of a distal end of an alternative medical device, according to aspects of this disclosure.

### DETAILED DESCRIPTION

Aspects of the disclosure include devices and methods for the at least partial aerosolization of an agent, including multi-part agents, and delivery of the at least partially aerosolized agent to a treatment site within a subject (e.g., patient). Current devices and methods for delivery of aerosolized agents are limited. Application of agents to the treatment sites may be challenging, for example, due to the fact that the treatment sites may not be smooth and/or difficult to reach. Additional challenges include poor, or reduced, visibility, particularly during and/or after application of the agent. In aspects, applying the agent unevenly on the treatment site may not fully protect and/or promote healing of the tissue. Application of an agent at a treatment site, for example, can protect those sites from further tissue degradation.

In some aspects of the disclosure, a medical device may include a multi-lumen sheath configured to deliver an agent (e.g., a hemostatic agent) within at least one lumen of the multi-lumen sheath and deliver a pressurized fluid (e.g., a gas) within at least one different lumen of the multi-lumen sheath. The agent and gas may remain separated throughout a length of the sheath. At a distal tip of the medical device, the agent may be at least partially aerosolized by the gas, and the aerosolized agent may be delivered to the treatment site (e.g., via at least one opening in the distal tip). Application of the aerosolized agent may promote healing, decrease procedural time, among other benefits.

The medical device may be movably disposed within a lumen of a scope (endoscope, bronchoscope, gastroscope, ureteroscope, duodenoscope, colonoscope, etc.), tube, sheath, or other insertion device that has been inserted into a body cavity or lumen, for example the gastrointestinal (GI) tract via a natural orifice. The orifice can be, for example, the nose, mouth, or anus, and the placement can be in any portion of the GI tract, including the esophagus, stomach, duodenum, large intestine, or small intestine. Delivery and placement also can be in other body lumens and/or organs reachable via the GI tract, any other natural opening or body tract, or bodily incision. In other aspects, the medical device may be inserted through the body cavity alone (e.g., without or alongside the scope, tube, or sheath).

Reference will now be made in detail to aspects of the disclosure, examples of which are illustrated in the accompanying drawings. Wherever possible, the same or similar reference numbers will be used through the drawings to refer to the same or like parts. Accordingly, between different embodiments, like numbers will be used to refer to like features, with “100” added to each numeral. Throughout various figures, the arrows “P” and “D” depict proximal and distal directions, respectively. The term “distal” refers to a portion farthest away from a user when introducing a device into a patient. By contrast, the term “proximal” refers to a portion closest to the user when placing the device into the subject. The systems and/or devices illustrated in the accompanying drawings may not be drawn to scale. Any of the devices disclosed herein may include any of the following features, additionally or alternatively, in any combination.

As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to systems and devices for performing various medical procedures and/or treating portions of a subject's anatomy. For example, systems and devices disclosed herein may be used to treat the large intestine (colon), small intestine, cecum, esophagus, any other portion of the GI tract, and/or any other suitable anatomy (collectively referred to herein as a “treatment site”). In particular, systems and devices disclosed herein may be used to deliver an agent to the treatment site. Various examples described herein include single-use or disposable medical devices that are, for example, comprised of one or more biocompatible materials.

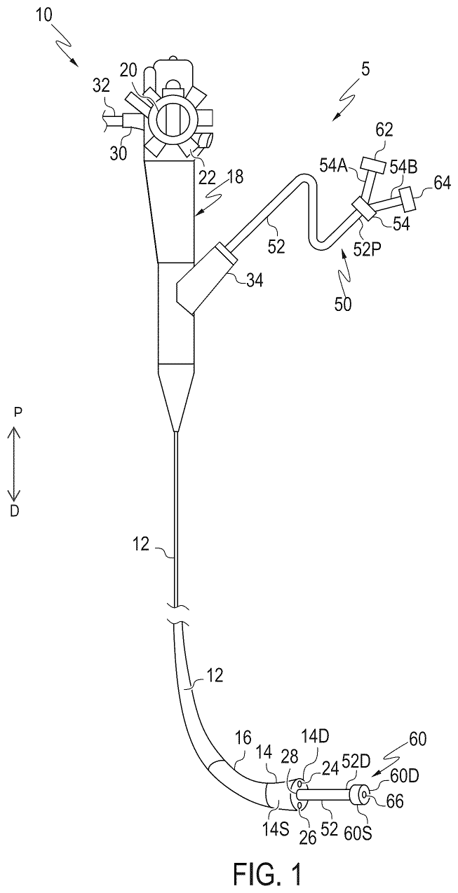

Referring to FIG. 1, a medical system 5 is shown. Medical system 5 may include a delivery device 10 and a medical device 50. Medical device 50 may be movably disposed within a lumen of delivery device 10. Medical device 50 may be used in conjunction with or independent of delivery device 10. Delivery device 10 may be, for example, a scope (e.g., endoscope, bronchoscope, gastroscope, ureteroscope, duodenoscope, colonoscope, etc.), tube, sheath, or insertion device configured for insertion into a body cavity or lumen of a subject.

In aspects, delivery device 10 may include a shaft 12 having an articulation portion 16 and a distal tip 14 at a distal end of delivery device 10. A handle 18 may be connected at a proximal end of shaft 12. Shaft 12 may include one or more lumens. In aspects, shaft 12 may be flexible, rigid, or semi-rigid.

Handle 18 may include one or more mechanisms configured to articulate or otherwise move articulation joint 16 in one or more directions, thereby moving distal tip 14 in one or more directions. For example, a plurality of actuating elements, such as cables or wires, may extend distally from a proximal end of delivery device 10 (e.g., such as handle 18), through one or more lumens of shaft 12, to articulation joint 16 and/or distal tip 14. For example, the actuating elements may be directly or indirectly coupled to first and second actuating devices 20, 22, which may control articulation of articulation joint 16 in multiple directions, such as up, down, left, and/or right.

Devices 20, 22 of handle 18, may be, for example, rotatable knobs that rotate about their axes to push/pull actuating elements extending through delivery device 10. Additional devices (e.g., knobs, buttons, levers, etc.) may be configured to control other aspects of delivery device 10. For example, additional devices may control aspects of distal tip 14, and/or aspects of elements attached to distal tip 14, such as an end effector. In some aspects, the additional devices may control up/down movement of an elevator (not shown) of distal tip 14.

Distal tip 14 may include an imaging device 24 (e.g., camera) and/or one or more lighting elements 26 (e.g., LEDs, optical fibers, etc.) disposed on a distalmost face 14D of distal tip 14. Although not shown, in some aspects, imaging device 24 and/or lighting elements 26 may, additionally or alternatively, be disposed on a side surface 14S of distal tip 14 (e.g., proximal to distalmost face 14D of distal tip 14). One or more electrical cables or wires may extend from the proximal end of delivery device 10 (e.g., handle 18), through shaft 12, and to imaging device 24 and/or lighting element(s) 26. The electrical cables or wires may provide electrical power and/or controls to imaging device 24, lighting element(s) 26, and/or other electrical devices in or on distal tip 14. In aspects, the electrical cables or wires may carry imaging signals from distal tip 14 proximally, for example, to be processed by a controller and/or displayed on a display. Distal tip 14 may further include one or more openings 28 disposed on distalmost face 14D. Opening(s) 28 may additionally or alternatively be disposed on side surface 14S of distal tip 14. Opening(s) 28 may be distal openings to one or more lumens (e.g., a working channel) extending through shaft 12.

A strain relief 30 may assist in attaching an umbilicus 32 to handle 18. Umbilicus 32 may be configured for introducing fluid, suction, and/or wiring for electronic components to handle 18. Umbilicus 32 may include a connector (e.g., for connecting umbilicus 32 to one or more of a controller, a display, etc., not shown).

Handle 18 may further include a port 34 for introducing and/or removing tools, fluids, devices, and/or other materials to and/or from the subject. For example, port 34 may be in fluid communication with one or more lumens (e.g., the working channel) of shaft 12 and distal tip 14. In aspects, port 34 may be configured to receive a portion of medical device 50. For example, a portion of medical device 50 may be inserted through port 34 and a lumen of shaft 12. A distal end of medical device 50 may be extended distally from distal tip 14, for example, via opening 28. Medical device 50 may be movable (e.g., translatable proximally and/or distally) within the lumen of delivery device 10, for example, controlling a length of medical device 50 that extends distally of opening 28.

Medical device 50 may include a sheath 52 having a proximal end 52P and a distal end 52D. A connector 54 may be coupled (e.g., directly or indirectly) to proximal end 52P of sheath 52. A distal tip 60 may be coupled (e.g., directly or indirectly) to distal end 52D of sheath 52. Sheath 52 may be comprised of one or more flexible, or bendable, materials. Described in more detail below, sheath 52 may include one or more lumens extending therethrough (e.g., from a proximal end 52P to a distal end 52D of sheath 52).

Connector 54 may be in fluid communication with the one or more lumens of sheath 52. In some aspects, connector 54 may resemble a Y-port having at least a first branch 54A and a second branch 54B. A first fluid source 62 and a second fluid source 64 may be directly or indirectly coupled to connector 54. For example, first fluid source 62 may be coupled to connector 54 via first branch 54A. Second fluid source 64 may be coupled to connector 54 via second branch 54B. Connector 54 may include additional branches to accommodate additional fluid sources. In aspects, each fluid source 62, 64 may be in fluid communication with a single lumen of sheath 52. For example, first fluid source 62 may be in fluid communication with a first lumen of sheath 52, and second fluid source 64 may be in fluid communication with a second lumen of sheath 52, or vice versa. One or both fluid sources 62, 64 may be in fluid communication with additional lumens of sheath 52. As discussed in detail below, fluid sources 62, 64 may contain each contain one or more gels, liquids, gases, powders, treatment agents, etc.

In aspects, first fluid source 62 may be configured to deliver a gas (e.g., atmospheric air, carbon dioxide, oxygen, nitrogen, etc.) through the first lumen of sheath 52. For example, first fluid source 62 may be in the form of a gas canister, a syringe, an insufflator pump, or any other device configured to deliver a gas. In some aspects, first fluid source 62 may also be fluidly connected to delivery device 10. As will be described in greater detail below, the gas delivered from first fluid source 62 may help to aerosolize an agent upon delivery from distal tip 60 (e.g., an agent delivered via second fluid source 64).

Second fluid source 64 may be configured to deliver an agent (e.g., a hemostatic agent) in liquid form. In some aspects, second fluid source 64 may be in the form of a syringe or irrigation pump. The agent of second fluid source 64 may be a single- or multi-part agent. For example, to deliver a multi-part agent, a first part (e.g., Part A) of the multi-part agent may be mixed or combined with a second part (e.g., Part B) of the multi-part agent. The multi-part agent may be mixed or combined, for example, before, during, or after the parts are loaded into second fluid source 64.

In aspects, each part of the multi-part fluid may be delivered together or separately. For example, Part A of the multi-part fluid may be delivered at a same time as Part B of the multi-part fluid. Alternatively, the first part may be delivered before or after the second part has been delivered. In aspects, the multi-part fluid may be combined within a lumen of sheath 52, at the distal tip 60, and/or upon delivery from medical device 50.

Additional fluid sources (e.g., a third fluid source, a fourth fluid source, etc.) may be used to deliver additional parts of the multi-part fluid. The additional fluid sources may be in fluid communication with the first lumen, second lumen, and/or additional lumens of sheath 52.

Once each part of the multi-part agent comes in contact with the other, the parts may crosslink. The cross-linked structure of the resulting combination of Parts A and B may have enhanced hemostatic properties, e.g., compared to Parts A or B individually. In a non-limiting example, part A may be fibrinogen (e.g., lyophilized pooled human concentrate), and part B may be thrombin (e.g., of bovine or human origin). The biocompatible fibrinogen and thrombin mixture may also contain calcium salts. The multi-part agent may include additional parts (e.g., a third part, a fourth part, etc.).

In some aspects, delivery of the gas from first fluid source 62 and/or delivery of the agent from second fluid source 64 may be manually or electronically controlled. For example, a user may manually deliver the gas from first fluid source 62 and/or the agent from second fluid source 64, for example, using a syringe. In other aspects, a user may electronically deliver the gas from first fluid source 62 and/or fluid from second fluid source 64, for example, via activation of a controller or device.

Distal tip 60 may have a diameter greater than a diameter of sheath 52. A distal opening 66 may be disposed on a surface of distal tip 60, such as, for example, a distalmost surface 60D or a side surface 60S (e.g., a surface that is proximal to distalmost surface 60D). Aspects of exemplary configurations of distal tip 60 are described in further detail below. In particular, the fluid delivered from first fluid source 62 may aerosolize the fluid delivered from second fluid source 64, for example, at or within distal tip 60. For example, the aerosolized agent may be delivered to the treatment site via distal opening 66 of distal tip 60.

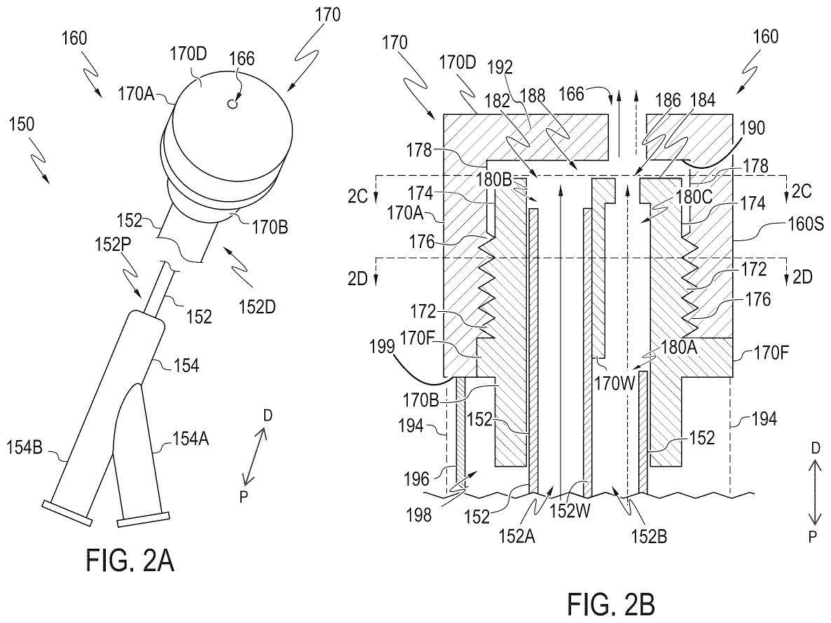

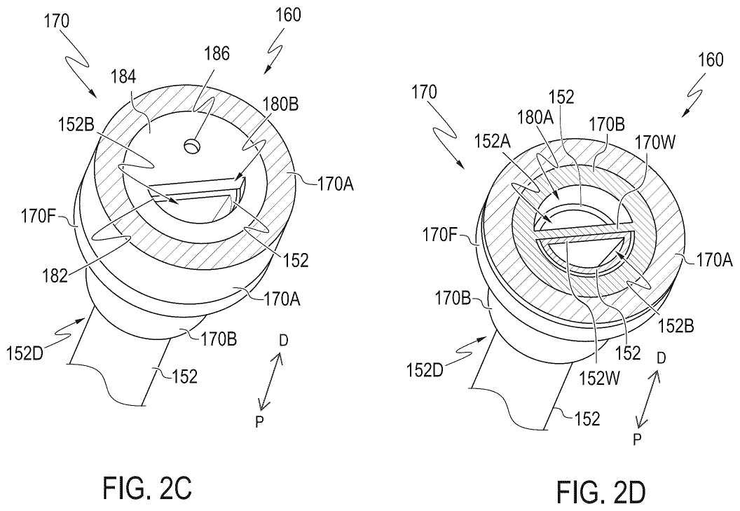

FIGS. 2A-2D illustrate various view of an exemplary medical device 150. For example, FIG. 2A illustrates a perspective view of medical device 150, including a distal portion 160; and FIG. 2B illustrates a longitudinal cross-sectional view of distal portion 160. FIG. 2C illustrates a first, transverse cross-sectional view of distal portion 160; and FIG. 2D illustrates a second, transverse cross-sectional view of distal portion 160 of medical device 150. Medical device 150 may be used alone or with delivery device 10 of system 5 of FIG. 1. For example, medical device 150 may have any or all of the same characteristics of medical device 50 of FIG. 1. Distal portion 160 may have any or all of the same characteristics of distal tip 60 of FIG. 1.

Referring primarily to FIG. 2A, medical device 150 may include a sheath 152 having a proximal end 152P and a distal portion 152D. Sheath 152 may have one or more lumens extending therethrough (e.g., from a proximal end 152P to a distal portion 152D of sheath 152). Proximal end 152P of sheath 152 may include a connector 154 in fluid communication with one or more lumens of sheath 152. A fluid source (e.g., fluid sources 62, 64 of FIG. 1) may be fluidly connected to each arm 154A, 154B of connector 154. Distal portion 160 may be coupled (directly or indirectly) to distal portion 152D of sheath 152.

Referring to FIGS. 2A-2D, distal portion 160 may include a cap assembly 170. Cap assembly 170 may include a first cap portion 170A and a second cap portion 170B. In aspects, at least a portion of first cap portion 170A may be distal relative to second cap portion 170B. First cap portion 170A may receive a distal portion of second cap portion 170B (as shown in FIG. 2B). In some aspects, first cap portion 170A may be unmovably fixed to second cap portion 170B. For example, first cap portion 170A may be fixed to second cap portion 170B using an adhesive, a press-fit, a friction-fit, a snap-fit, etc., or any combination thereof. In aspects, first cap portion 170A and second cap portion 170B may be integrally formed.

In other aspects, first cap portion 170A may be movably fixed to second cap portion 170B. Second cap portion 170B may include a plurality of threads 172 (FIG. 2B) disposed along at least a portion of an outward-facing surface 174 of second cap portion 170B. A plurality of receiving threads 176 may be disposed on an inward surface 178 of first cap portion 170A. The plurality of receiving threads 176 of first cap portion 170A may be configured to receive the plurality of threads 172 of second cap portion 170B. For example, when first cap portion 170A is rotated relative to second cap portion 170B, first cap portion 170A may be coupled, or secured, to second cap portion 170B via the plurality of threads 172 and the plurality of receiving threads 176, for example, similar to a screw cap.

Second cap portion 170B may include one or more flanges 170F. Flange(s) 170F may be configured to prevent first cap portion 170A from moving proximally past flange(s) 170F. One or more flange(s) 170F may extend radially outward (e.g., relative to a longitudinal axis of distal portion 160), for example, and define an outer, side surface 160S of distal portion 160. Additionally or alternatively, a portion of first cap portion 170A may extend proximally over flange(s) 170F.

Second cap portion 170B may be configured to receive at least a portion of distal portion 152D of sheath 152. For example, second cap portion 170B may define a first space 180A, a second space 180B, and a third space 180C. First space 180A may be proximal of second space 180B and third space 180C. Second space 180B may be adjacent to third space 180C. A dividing wall 170W of second cap portion 170B may divide second space 180B from third space 180C. In aspects, dividing wall 170W may be configured to provide structural support to a distal portion of first lumen 152A of sheath 152 (e.g., a portion of first lumen 152A that extends distally relative to second lumen 152B of sheath 152). First space 180A may encompass an entire cross-section of a proximal portion of second cap portion 170B. For example, first space 180A may be configured to receive an entire diameter of distal portion 152D of sheath 152. In these aspects, the cross-sectional shape of first space 180A may be complementarily shaped to receive distal portion 152D of sheath 152. For example, a cross-sectional shape of first space 180A may be generally circular or ovular.

First lumen 152A of sheath 152 may extend through first space 180A and distally into second space 180B. A distal end of first lumen 152A may extend distally relative to a distal end of second lumen 152B of sheath 152. For example, a distalmost end of second lumen 152B may be proximal to a distalmost end of first lumen 152A. A distal end of a second lumen 152B of sheath 152 may be disposed within first space 180A. For example, the distal end of second lumen 152B of sheath 152 may terminate proximally of third space 180C. In other aspects, the distal end of second lumen 152B of sheath 152 may extend at least partially distally into third space 180C.

A central wall 152W of sheath 152 may divide first lumen 152A from second lumen 152B. Central wall 152W of sheath 152 may extend an entire length of sheath 152. In aspects, central wall 152W may be integrally formed with an outermost wall defining sheath 152. First lumen 152A and second lumen 152B may be a same size and/or shape. In other aspects, first lumen 152A and second lumen 152B may each be different sizes and/or shapes. Although sheath 152 is illustrated as having a first lumen 152A and a second lumen 152B, sheath 152 may include any number of lumens (e.g., three, four, etc.). Accordingly, second cap portion 170B may include one or more additional spaces (e.g., a fourth space, a fifth space, etc.) that is/are configure to receive one or more lumens of sheath 152. In other aspects, second space 180B and/or third space 180C may be configured to receive additional lumens of sheath 152.

Referring to FIGS. 2B and 2C, a distal end of second space 180B may include a first opening 182 disposed on a distalmost surface 184 of second cap portion 170B. First opening 182 may have a same diameter as an inner diameter of second space 180B. In aspects, first opening 182 may have a diameter that is at least equal to an inner diameter of second space 180B. Shown in FIG. 2C, a cross-sectional shape of first opening 182 may be semi-circular or semi-ovular. In aspects, a cross-sectional shape of first opening 182 may be the same as a cross-sectional shape of second space 180B (e.g., proximal of first opening 182). In aspects, first lumen 152A may have a same or similar cross-sectional shape as first opening 182 and/or second space 180B.

Shown more clearly in FIG. 2D, a portion of second space 180B and third space 180C (e.g., proximal of first opening 182 and second opening 186) may each have semi-circular cross-sectional shapes. For example, a longest arc of each of second space 180B and third space 180C may be equal. In other aspects, a cross-sectional shape of second space 180B may be greater than a cross-sectional shape of third space 180C, or vice versa. In other words, a longest arc of second space 180B may be greater than a longest arc of third space 1800, or vice versa. When observed together, the cross-sectional shapes of second space 180B and third space 180C may define a full circular, or ovular, cross-sectional shape of second cap portion 170B.

A distal end of third space 180C may include a second opening 186 disposed on a distalmost surface 184 of second cap portion 170B. In aspects, a cross-sectional shape of second opening 186 may be circular, or ovular. The cross-sectional shape of second opening 186 may be different than a cross-sectional shape of third space 180C (e.g., proximal of second opening 186). Second opening 186 may have a smaller diameter as compared to an inner diameter of third space 180C.

A cavity 188 may be defined between first cap portion 170A and second cap portion 170B. For example, cavity 188 may be defined between distalmost surface 184 of second cap portion 170B and a proximally-facing surface 190 of first cap portion 170A. In these aspects, first opening 182 may permit fluid flow from first lumen 152A and into cavity 188. Second opening 186 may also permit fluid flow from second lumen 152B and into cavity 188. Distal opening 166 may permit fluid flow out of cavity 188. In aspects, distal opening 166 may be aligned (e.g., concentric about a center point of each of distal opening 166 and second opening 186) with second opening 186 of second cap portion 178B. A diameter of distal opening 166 may be larger than a diameter of second opening 186. A distal wall 192 of first cap portion 160A may extend over first opening 182. For example, distal wall 192 of first cap portion 160A may assist in diverting fluid from first lumen 152A towards distal opening 166.

In aspects, cavity 188 may provide a space for the fluid from first lumen 152A and the gas from second lumen 152B to at least partially mix or combine. For example, during use, fluid from first lumen 152A may flow from first lumen 152A, through first opening 182, into cavity 188. Distal wall 192 may help to divert the fluid to flow towards distal opening 166. As the gas flows from second lumen 152B, though third space 1800, second opening 186, and into cavity 188, the fluid within cavity 188 becomes at least partially aerosolized. For example, aerosolized particles of the fluid may be delivered from distal portion 160, through distal opening 166. The aerosolized fluid may then be applied to target tissue, for example, at the treatment site.

A length, and thereby, a volume, of cavity 188 may change, for example, depending on the characteristics of the fluids, the characteristics of the aerosol being delivered. For example, for highly viscous fluids, a cross sectional height of cavity 188 may be smaller to facilitate a smaller interface area (e.g., space or volume) between the fluid and the gas to enable effective shearing of the viscous fluid required for atomization, and/or to achieve smaller aerosolized particles. For lower viscosity materials, cavity 188 may be larger, as shearing lower viscosity materials may be easier to accomplish. In some aspects, having a larger interface area (e.g., space or volume) between the fluid and the gas may enable higher aerosol densities.

To increase and/or decrease the length or volume of cavity 188, first cap portion 170A may be rotated relative to second cap portion 170B. For example, first cap portion 170A may be rotated in a first direction to increase or decrease the volume of cavity 188. First cap portion 170A may be rotated in a second direction (e.g., opposite the first direction) to decrease the volume of cavity 188.

Referring back to FIG. 2B, in some aspects, medical device 150 may include an outer sheath 194. For example, sheath 152 may be an inner sheath within one or more lumens of outer sheath 194. A proximal end of second cap portion 170B may extend proximally into a lumen 198 of outer sheath 194. In some aspects, a distalmost end of outer sheath 194 may abut and/or be fixed to flange(s) 170F of second cap portion 170B. A control member 196 may extend proximally through lumen 198 of outer sheath 194. Alternatively, control member 196 may extend adjacent to or along an outer surface or circumference of outer sheath 194. Control member 196 may be fixed to and extend proximally from a proximal face 199 of first cap portion 170A.

In aspects, control member 196 may be a wire, a cord, a cable, a bundle of wires, a sheath, a tube, etc., configured to assist a user with rotating first cap portion 170A relative to second cap portion 170B and/or with controlling a position of distal portion 160. In these aspects, control member 196 may be configured to assist with rotating first cap portion 170A relative to second cap portion 170B, for example, when distal portion 160 is in situ. In these aspects, control member 196 may extend proximally to and/or past connector 154 of medical device 150. In some aspects, control member 196 may be coupled to an actuator and/or a handle of medical device 150.

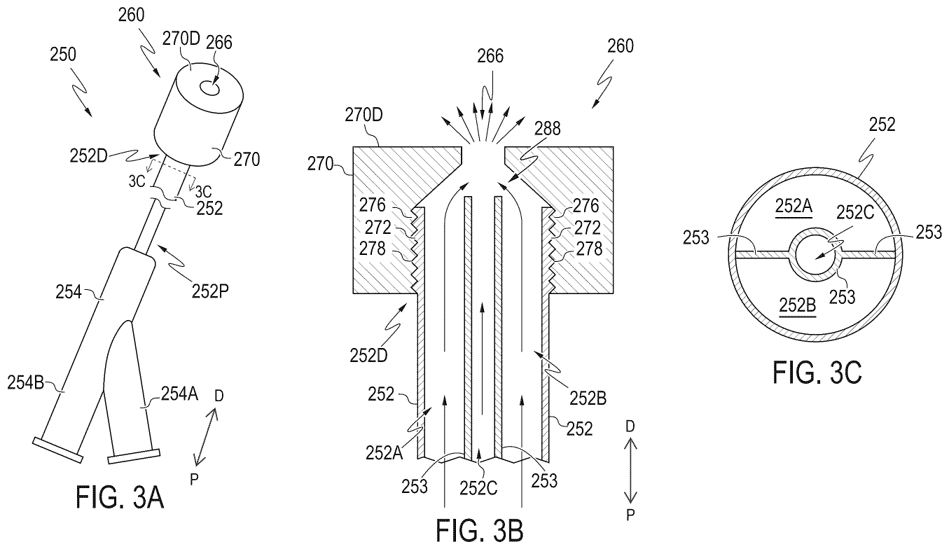

FIGS. 3A-3C illustrate various views of an alternative medical device 250. For example, FIG. 3A illustrates a perspective view of medical device 250. FIG. 3B illustrates a longitudinal cross-sectional view of a distal portion 260 of medical device 250; and FIG. 3C illustrates a transverse cross-sectional view of distal portion 260. Medical device 250 may be used alone or with delivery device 10 of system 5 (FIG. 1). Medical device 250 may have any or all of the same characteristics of medical device 50 and/or medical device 150, except as described below. For example, medical device 250 may include a sheath 252 having a proximal end 252P and a distal end 252D. A connector 254 may be coupled to proximal end 252P. A first branch 254A of connector 254 may be fluidly coupled to a first fluid source (e.g., first fluid source 62 of FIG. 1). A second branch 254B of connector 254 may be fluidly coupled to a second fluid source (e.g., second fluid source 64 of FIG. 1).

Distal portion 260 of medical device 250 may include a cap 270. Cap 270 may be coupled to distal end 252D of sheath 252, for example, using an adhesive, a press-fit, a friction-fit, a snap-fit, etc., or any combination thereof. In aspects, cap 270 and distal end 252D of sheath 252 may be integrally formed. In other aspects, cap 270 may be movably coupled to distal end 252D of sheath 252. For example, a plurality of threads 272 (FIG. 3B) may be disposed along a portion of distal end 252D of sheath 252. A plurality of receiving threads 276 may be disposed on an inward surface 278 of cap 270. The plurality of receiving threads 276 may be configured to receive the plurality of threads 272 of sheath 252. For example, when cap 270 is rotated relative to sheath 252, cap 270 may be coupled, or secured, to sheath 252 via the plurality of threads 272 and the plurality of receiving threads 276, for example, similar to a screw cap.

Sheath 252 may include a plurality of lumens. For example, shown in FIGS. 3B and 3C, sheath 252 may include a first lumen 252A, a second lumen 252B, and a third lumen 252C. First lumen 252A and second lumen 252B may each at least partially surround third lumen 252C. In some aspects, a cross-sectional shape of each of first lumen 252A and second lumen 252B may be C-shaped or U-shaped. In these aspects, a perimeter of each of first lumen 252A and second lumen 252B may include a minor arc and a major arc. A cross-sectional shape of third lumen 252C may be a circle or an oval. Together, first lumen 252A and second lumen 252B may form a ring around third lumen 252C. The minor arcs of first lumen 252A and second lumen 252B may form at least part of a perimeter of third lumen 252C. First lumen 252A, second lumen 252B, and third lumen 252C may be separated by one or more walls 253. In aspects, wall(s) 253 may support third lumen 252C. First lumen 252A, second lumen 252B, and third lumen 252C may be configured to receive a fluid (e.g., a liquid and/or a gas). In aspects, a distalmost end of third lumen 252C may extend distally relative to a distalmost end of first lumen 252A and second lumen 252B.

A cavity 288 may be defined between a distalmost end of sheath 252 and internal walls of cap 270. In aspects, a volume of cavity 288 may be increased or decreased. For example, by rotating cap 270 in a first or second direction relative to sheath 252, the volume of cavity 288 may increase or decrease. In aspects, internal walls of cap 270 may be angled towards a distal opening 266 of cap 270, for example, to facilitate fluid flow towards distal opening 266. Distal opening 266 may be disposed on a distal face 270D of cap 270. Distal opening 266 may be aligned with fifth lumen 352F. For example, distal opening 266 and fifth lumen 352F may be concentric about a longitudinal axis extending through distal opening 266 and third lumen 252C.

During use, a multi-part agent may be delivered. For example, a first part (e.g., Part A) of a multi-part agent may be delivered via first lumen 252A. A second part (e.g., Part B) of a multi-part agent may be delivered via second lumen 252B. Part A and Part B may combine, or mix, within cavity 288. In some aspects, Part A and Part B may crosslink within cavity 288. A gas may be delivered via third lumen 252C. The gas from third lumen 252C may help to aerosolize the combined parts (e.g., Part A and Part B) contained within cavity 288. The at least partially aerosolized combination or mixture may then be delivered via distal opening 266.

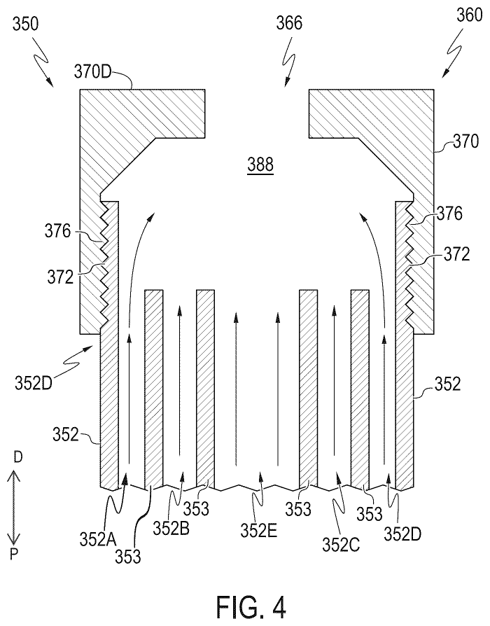

FIG. 4 illustrates a longitudinal cross-sectional view of a distal portion 360 of a medical device 350. Medical device 350 may be used alone or with delivery device 10 of system 5 (FIG. 1). Medical device 350 may have any or all of the same characteristics of medical device 50, medical device 150, and/or medical device 250, except as described below. For example, medical device 350 may include a sheath 352 having a distal end 352D. Although not shown, a proximal end of sheath 352 may include a connector (e.g., similar to any one of connectors 54, 154, and/or 254). In these aspects, one or more lumens of sheath 352 may be fluidly connected one or more fluid sources (e.g., fluid sources 62, 64 of FIG. 1).

Sheath 352 may include a plurality of lumens extending from the proximal end to distal end 352D. For example, sheath 352 may include a first lumen 352A, a second lumen 352B, a third lumen 352C, a fourth lumen 352E, and a fifth lumen 352F. Each lumen (e.g., lumen 352A, 352B, 352C, 352E, 352F) may be separated by one or more dividing walls 353. In aspects, one or more lumens may have a same cross-sectional size and/or shape. In other aspects, one or more lumens may have a different cross-sectional size and/or shape.

Distal portion 360 of medical device 350 may include a cap 370 fixed to distal end 352D of sheath 352. Cap 370 may be coupled to distal end 352D of sheath 352, for example, using an adhesive, a press-fit, a friction-fit, a snap-fit, etc., or any combination thereof. In aspects, cap 370 and distal end 352D of sheath 352 may be integrally formed. In other aspects, cap 370 may be movably coupled to distal end 352D of sheath 352. For example, a plurality of threads 372 (FIG. 3B) may be disposed along a portion of distal end 352D of sheath 352. A plurality of receiving threads 376 may be disposed on an inward surface of cap 370. The plurality of receiving threads 376 may be configured to receive the plurality of threads 372 of sheath 352. For example, when cap 370 is rotated relative to sheath 352, cap 370 may be coupled, or secured, to sheath 352 via the plurality of threads 372 and the plurality of receiving threads 376, e.g., similar to a screw cap.

A cavity 388 may be defined between a distalmost end of sheath 352 and internal walls of cap 370. In aspects, a volume of cavity 388 may be increased or decreased. For example, by rotating cap 370 relative to sheath 352, the volume of cavity 388 may increase or decrease. Additionally or alternatively, one or more dividing walls 353 may be extended distally or retracted proximally to increase or decrease the volume of cavity 388. In aspects, internal walls of cap 370 may be angled towards a distal opening 366 of cap 370, for example, to facilitate fluid flow towards distal opening 366. Distal opening 366 may be disposed on a distal face 370D of cap 270. In some aspects, distal opening 366 may be aligned with fifth lumen 352F. For example, distal opening 366 and a distal opening of fifth lumen 352F may be concentric about a longitudinal axis extending through both distal opening 366 and fifth lumen 352F . . .

Medical device 350 may be used similarly to medical device 50, 150, and/or 250. For example, medical device 350 may be used to deliver a multi-part agent. For example, a first part (e.g., Part A) of a multi-part agent may be delivered via first lumen 352A. A second part (e.g., Part B) may be delivered via second lumen 352B. A third part (e.g., Part C) and a fourth part (e.g., Part D) may be delivered via third lumen 352C and fourth lumen 352E, respectively. Each part (e.g., Parts A-D) may combine or mix within cavity 397. A gas may be delivered via fifth lumen 352F. Accordingly, the gas may at least partially aerosolize the combined mixture. The at least partially aerosolized combination or mixture may then be delivered via distal opening 366.

Aspects of any of the aforementioned systems and devices may be used in any combination or arrangement. For example, cap 270 of FIGS. 3A-3C may be used in place of cap assembly 170 of FIGS. 2A-2B, or vice versa. Similarly, cap assembly 170 may be fixed to distal end 352D of sheath 352 (FIG. 4). Additional combinations or arrangements are also contemplated herein.

Each of the aforementioned systems and devices may be used to protect and/or treat treatment sites by delivering an aerosolized agent(s) (a gel, a powder, or one or more other prophylactic agents) to the treatment site. By providing a medical device with multiple lumens for delivering an agent in aerosol form, known problems associated with uneven application of the agent to the treatment site and/or other aspects of invasive surgical procedures may be reduced or avoided. For example, the delivery of one or more agents in aerosol form may help to form a smooth (e.g., conformal) and/or even layer of agent (e.g., adhesive) across a surface of the treatment site, which may help to protect the treatment site and/or promote healing.

Aspects of this material may help to apply the agent(s) in a thin layer, which may help facilitate material (e.g., gel) properties, and/or result in a lower profile cover or layer over the treatment site, which may help to reduce the risk of the applied agent(s) becoming dislodged, being sheared, or otherwise separating from the treatment site (e.g., from other fluids and/or solids adjacent to the treatment site, for example, the GI tract). For example, aspects of this disclosure may help to form one or more layers of agent(s) to help minimize delayed bleeds, potential perforations, stricture formations, etc. Accordingly, physicians or other users may reduce the overall procedure time, increase efficiency of procedures, and/or avoid unnecessary harm to a subject's body caused by limited ability of other tools/devices to treat perforations, post-surgical leaks, and/or other wounds that might result from endoscopic and open surgical procedures of a body lumen (e.g., the GI tract).

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only, with a true scope of the disclosure being indicated by the following claims and their equivalents.

We claim:

1. A medical device, comprising:
a sheath having a proximal end, a distal end, a first lumen extending within the sheath from the proximal end to the distal end, and a second lumen extending within the sheath from the proximal end to the distal end;
a cap coupled to the distal end of the sheath, wherein the cap defines a cavity between the distal end of the sheath and a distal wall of the cap, wherein a distal opening extends through the distal wall of the cap; and
a connector at the proximal end of the sheath, wherein the connector is configured to provide fluid communication between: (1) a first fluid source and the first lumen, and (2) a second fluid source and the second lumen,
wherein, upon delivery of a fluid from the first lumen into the cavity and a gas from the second lumen into the cavity, an at least partially aerosolized fluid is delivered via the distal opening.

2. The medical device of claim 1, wherein the distal opening of the cap is aligned with a distal end of the second lumen.

3. The medical device of claim 1, wherein the cap is comprised of a first cap portion and a second cap portion, wherein the first cap portion is configured to receive at least a portion of the second cap portion.

4. The medical device of claim 3, wherein the second cap portion includes at least one flange extending radially outward, wherein the at least one flange is configured to prevent proximal movement of the first cap portion.

5. The medical device of claim 3, wherein the distal opening is a first distal opening that extends through the distal wall of the first cap portion, wherein the second cap portion defines a second distal opening that is proximal of the first distal opening, and wherein a diameter of the second distal opening is smaller than a diameter of the first distal opening.

6. The medical device of claim 3, further comprising a control member extending proximally from a proximal end of the first cap portion.

7. The medical device of claim 6, wherein the sheath is an inner sheath disposed within a lumen of an outer sheath, wherein the control member extends proximally into the lumen of the outer sheath.

8. The medical device of claim 1, wherein the fluid is a hemostatic agent.

9. The medical device of claim 1, wherein the gas is at least one of carbon dioxide, oxygen, nitrogen, or atmospheric air.

10. The medical device of claim 1, wherein a plurality of threads is disposed on a distal end of the sheath, and wherein the cap includes a plurality of receiving threads configured to receive the plurality of threads of the sheath to secure the cap to the sheath.

11. The medical device of claim 10, wherein rotation of the cap in a first direction relative to the sheath increases a volume of the cavity, wherein rotation of the cap in a second direction relative to the sheath decreases the volume of the cavity, and wherein the second direction is opposite to the first direction.

12. The medical device of claim 1, wherein a distal end of the first lumen is distal relative to a distal end of the second lumen.

13. The medical device of claim 1, wherein a portion of the distal wall of the cap is spaced away from and aligned with a distal opening of the first lumen.

14. The medical device of claim 1, wherein a cross-sectional shape of each of the first lumen and the second lumen is C-shaped, and wherein the sheath further defines a third lumen disposed between the first lumen and the second lumen.

15. The medical device of claim 1, wherein a diameter of the cap is greater than a diameter of the sheath.

16. An insertion portion of a medical device, comprising:
a sheath defining a first lumen and a second lumen; and
a cap configured to be coupled to a distal end of the sheath, wherein the cap includes a distal wall having a distal opening extending therethrough, wherein a cavity is defined between the distal wall and a distal end of each of the first lumen and the second lumen,
wherein, upon delivery of a fluid from the first lumen into the cavity and upon delivery of a gas from the second lumen into the cavity, an aerosol is delivered via the distal opening of the cap.

17. The insertion portion of claim 16, wherein the distal opening of the cap is at least partially aligned with a distal opening of the second lumen.

18. The insertion portion of claim 16, wherein rotation of the cap relative to the sheath in a first direction is configured to increase a volume of the cavity, wherein rotation of the cap relative to the sheath in a second direction is configured to decrease a volume of the cavity, and wherein the second direction is opposite to the first direction.

19. A medical device, comprising:
a sheath defining a first lumen and a second lumen,
a connector fixed to a proximal end of the sheath and fluidly connected to the first lumen and the second lumen; and
a cap fixed to a distal end of the sheath, the cap defining a cavity and a distal opening, wherein when the cap is coupled to a distal end of the sheath, the distal opening is at least partially aligned with a distal end of the second lumen,
wherein, upon: (1) delivery of a fluid through the first lumen and into the cavity and (2) delivery of a gas through the second lumen and into the cavity, an aerosol is delivered through the distal opening of the cap.

20. The medical device of claim 19, wherein rotation of the cap relative to the sheath in a first direction is configured to increase a volume of the cavity, wherein rotation of the cap relative to the sheath in a second direction is configured to decrease a volume of the cavity, and wherein the second direction is opposite to the first direction.
